(19) United States
(12) Patent Application Publication
(10) Pub. No.: US 2007/0244413 A1
(43) Pub. Date: Oct. 18, 2007

(54) Medical guidewire tip construction

(75) Inventor: James F. Biggins, Waltham, MA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/403,043

(22) Filed: Apr. 12, 2006

(51) Int. Cl. A61M 25/00

(52) U.S. Cl. 600/585

(57) ABSTRACT

A medical guidewire having a core-to-tip construction that includes a core wire region surrounded by a flexible coil. The core wire having a distal tip segment that includes a proximal flat drop axially separated from a distal flat drop by a cylindrical or frusto-conical linking portion. The proximal and distal flat drops each having a pair of parallel planar surfaces, wherein the planar surfaces of the proximal flat drop are at an angle to the planar surfaces of the distal flat drop. The tip construction provides improved flexibility while maintaining columnar strength and providing excellent torsional characteristics.

### FIELD OF THE INVENTION

The invention relates to medical guidewires used to assist in the placement of catheters in body lumens and, particularly, to an improved tip structure for such guidewires.

### BACKGROUND OF THE INVENTION

Medical guidewires are used in numerous catheterization procedures as an aid to placement of a catheter at a selected site within a body lumen. The catheter is constructed to perform a particular procedure at that internal site. Among the more common uses of guidewires is in the catheterization of blood vessels for diagnostic or therapeutic purposes. In such a vascular catheterization procedure, the guidewire is inserted, usually percutaneously, into one of the patient's blood vessels and is manipulated and advanced through the branches of the vascular system to the target site. The catheter is then threaded over and advanced along the guidewire, with the guidewire serving to guide the catheter directly to the target site.

A number of catheterization procedures are performed with respect to the coronary arteries. In one such procedure for diagnostic purposes, an angiographic catheter is advanced through the vasculature to the coronary arteries. A radiopaque contrast liquid then is injected through the angiographic catheter into the coronary arteries under X-ray fluoroscopy, so that the anatomy of the patient's coronary arteries may be visually observed. Once the condition of the coronary anatomy has been determined, the physician may perform additional catheterization procedures, including percutaneous transluminal coronary angioplasty (PTCA), in which a balloon catheter or other angioplasty catheter is advanced into the coronary arteries to widen an obstructed portion, i.e., a stenosis, of the artery.

In a typical PTCA procedure, an angioplasty catheter, which may be in the form of an elongate flexible shaft with an inflatable balloon at its distal end, is advanced from the percutaneous puncture site in the patient's femoral artery through the patient's arteries toward the heart and into the coronary arteries. The catheter is guided to the target site of the obstruction by use of a slender guidewire, which initially is advanced into and manipulated through the coronary arteries in advance of the dilatation catheter. Once the distal region of the guidewire is in place within the obstruction, the catheter is advanced over the guidewire to place its balloon within the obstruction. The balloon is inflated to dilate the obstructed portion of the artery, thereby enlarging the flow area through the artery.

Guidewires used with PTCA catheters may be extremely slender, in the order of 0.25 to 0.46 mm (0.010 to 0.018 inches) in diameter, but nevertheless must be capable of transmitting rotation from the guidewire proximal end to the distal end so that a clinician may controllably steer the guidewire through the branches of the patient's arteries and manipulate it to the target site in the intended coronary artery. Additionally, the distal region of the guidewire must be sufficiently flexible to pass through sharply curved tortuous coronary anatomy, as well as to provide a sufficiently soft, distal tip that will not injure the artery. In addition, a guidewire must have sufficient column strength so that it can be pushed without buckling.

A guidewire configuration used in angioplasty is illustrated in U.S. Pat. No. 4,545,390 to Leary. Such a wire includes an elongate flexible shaft, typically formed from stainless steel, having a tapered distal region and a helical coil mounted to and about the tapered distal region. The generally tapering distal region of the shaft acts as a core for the coil and results in a guidewire having a distal region of increasing flexibility that is adapted to follow the contours of the vascular anatomy while still being capable of transmitting rotation from the proximal end of the guidewire to the distal end, so that the physician can controllably steer the guidewire through the patient's blood vessels.

Performance characteristics of the guidewire are affected by the construction of the guidewire distal tip. For example, in one type of tip construction, the tapering core wire extends fully through the helical coil to the distal tip of the coil and is attached directly to a smoothly rounded tip weld at the distal tip of the coil. Such a construction, referred to as a core-to-tip construction, typically results in a relatively stiff tip particularly suited for use through tight stenosis. In addition to a high degree of column strength, such a tip also displays excellent torsional characteristics.

In another type of tip construction, the tapered core wire terminates short of the tip weld. In such a construction, a very thin metallic ribbon may be attached between a distal end of the core wire and the smoothly rounded tip weld at the distal tip of the coil. The ribbon serves as a safety element to maintain the connection between the core wire and the distal tip weld in the event of coil breakage. It also serves as a shaping ribbon for receiving and retaining a bend or curve to maintain the guidewire distal segment in a bent configuration, as may be desirable when manipulating and steering the guidewire subselectively into vessel side branches. Additionally, by terminating the core wire short of the tip weld, the segment of the helical coil between the distal end of the core wire and the tip weld is very flexible or “floppy.” The so-called floppy (ribbon) tip is desirable in situations where the vasculature is highly tortuous and in which the guidewire distal segment must be capable of conforming to and following the tortuous anatomy with minimal trauma to the blood vessel.

In another type of tip construction, known as a “flat-drop,” a distalmost segment of the core wire is hammered or forged into a parallel or tapering flat segment to serve the same function as the safety/shaping ribbon but as an integral, unitary piece with the core wire. The tip of the flat-dropped segment is attached to the smoothly rounded tip weld at the distal tip of the coil.

Although each of the above-described tip constructions has its advantages, each also presents some compromises and difficulties. Although the construction in which the core extends fully to, and is attached to the tip weld, i.e., a core-to-tip construction, is particularly suited for crossing a very tight stenosis, it may be unsuitable in those instances where a more tortuous anatomy with a less restrictive stenosis is encountered. Among the difficulties presented with a ribbon tip construction is that the relatively low bending stiffness of the distal tip sometimes permits the ribbon and the surrounding coil to prolapse, that is, to fold back on itself. The safety/shaping ribbon also provides lower tensile strength than a core-to-tip construction. Ribbon tip construction also provides reduced torsional stiffness, which can diminish torque transmission, i.e., steering to the guidewire tip, while increasing the number of rotations-to-failure.

What is needed is a tip construction for a guidewire with sufficient flexibility to negotiate a tortuous anatomy while maintaining sufficient column strength to transmit torque and facilitate steering.

### BRIEF SUMMARY OF THE INVENTION

An embodiment according to the present invention is an intravascular guidewire for use in guiding a catheter through a body lumen. The guidewire includes an elongate shaft having a reduced-diameter distal region that defines a cylindrical core wire. The core wire has a distal tip segment that includes a proximal flat drop spaced from a distal flat drop by a cylindrical linking portion. The core wire distal region of the guidewire shaft is surrounded by a flexible coil. In an embodiment, a planar surface of the proximal flat drop of the core wire may be substantially perpendicular to a planar surface of the distal flat drop of the core wire.

In an embodiment, the guidewire shaft is a unitary structure having a tapered core wire distal region. In various embodiments, a length, width and/or thickness dimension of the proximal and distal core wire flat drops may be the same or varied. In an embodiment, a thickness of at least one of the proximal and distal flat drops is tapered.

In another embodiment, an intravascular guidewire according to the present invention includes an elongate shaft having a reduced-diameter core wire region. The core wire region has a distal tip segment that includes a plurality of flat drops spaced from each other by cylindrical linking portions. Planar surfaces of adjacent flat drops are at an angle to each other, such that the surfaces are not in the same plane. In various embodiments, the core wire segment may include a planar surface of at least one flat drop that is substantially perpendicular to a planar surface of at least one other flat drop and/or one or more cylindrical linking portions may be tapered. A flexible coil surrounds and is attached to at least the core wire region of the guidewire shaft.

### BRIEF DESCRIPTION OF DRAWINGS

The foregoing and other features and advantages of the invention will be apparent from the following description of the invention as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 is a side view of a guidewire in accordance with an embodiment of the present invention.

FIG. 2 is a partial cross-sectional view of a distal region of the guidewire of FIG. 1.

FIG. 3 is a side view of a distalmost section of the guidewire illustrated in the embodiment of FIG. 2.

FIG. 4 is a top plan view of the guidewire section illustrated in FIG. 3.

FIG. 5 is a side view of the core wire tip segment illustrated in the embodiment of FIG. 3.

FIG. 6 is a top plan view of the core wire tip segment illustrated in FIG. 5.

### DETAILED DESCRIPTION OF THE INVENTION

Specific embodiments of the present invention are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

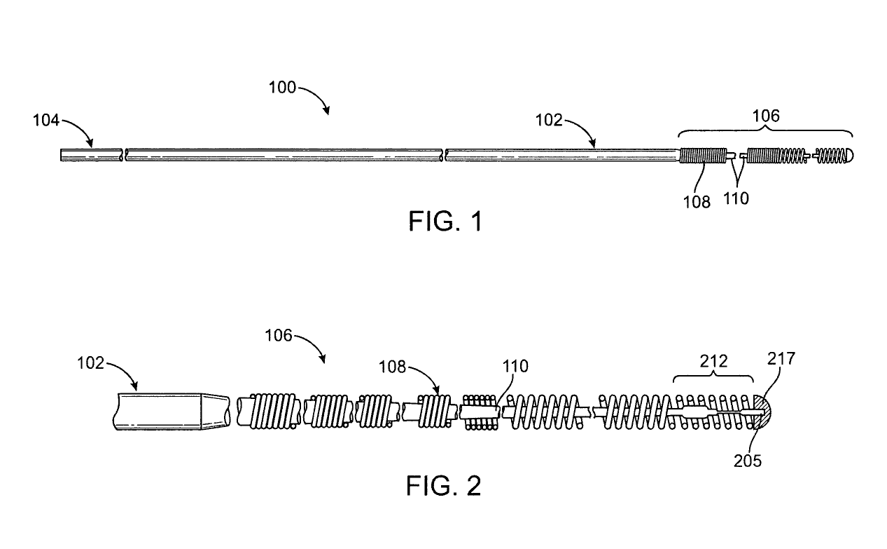

FIGS. 1 and 2 illustrate a side view of a guidewire 100 in accordance with an embodiment of the present invention. Guidewire 100 includes an elongate shaft 102 formed from an appropriate material, such as stainless steel, nitinol, an alloy of tungsten-rhenium, or, a work-hardenable cobalt chromium superalloy such as 35NLT. Shaft 102 has a proximal end 104, a distal end 205, and a distal region 106. Proximal end 104 of shaft 102 may be provided with a tubular socket (not shown), or may be otherwise adapted for connection with a guidewire extension, as such guidewire extension systems would be apparent to one of ordinary skill in the art. A flexible helical coil 108 surrounds distal region 106 of shaft 102. As would be apparent to one of ordinary skill in the relevant art, coil 108 may comprise a flexible tubular sheath instead of, or in combination with a coiled filament. A proximal end of coil 108 may be attached, for example by soldering, brazing, or by adhesive at a proximal end of distal segment 106 and may also be attached at a point or points along its length within distal segment 106. A distal end of coil 108 is secured to distal end 205 of shaft 102 within a hemispherical tip weld 217. As would be apparent to one of ordinary skill in the art, tip weld 217 may comprise a weld with or without added filler material, or a joint including braze, solder or adhesive. In an embodiment, flexible coil 108 may be formed from a radiopaque alloy, such as a stainless steel-platinum, gold-platinum or a platinum-tungsten alloy.

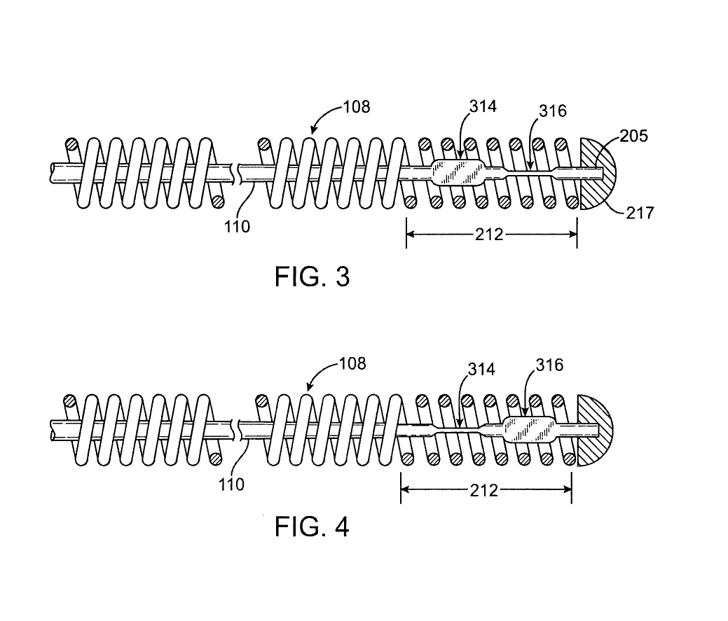

Distal region 106 of shaft 102 may include a continuous or stepped taper, for example, as disclosed in U.S. Pat. No. 4,922,924 to Gambale et al., which is incorporated by reference herein in its entirety. As illustrated in the embodiment of FIGS. 2-4, distal region 106 of shaft 102 includes a tapered core wire 110 having a distal tip segment 212. Core wire tip segment 212 is provided with proximal and distal flat drops 314, 316, which are axially-spaced flattened portions of core wire 110 that are wider than an adjacent outer diameter of core wire 110. As mentioned above, distal end 205 of core wire 110 is fixed within tip weld 217 at a distal tip of coil 108. In another embodiment, distal flat drop 316 of core wire 110 may extend to and be fixed within tip weld 217.

Elongate shaft 102 may be a unitary shaft from proximal end 104 to distal end 205, wherein distal region 106 of shaft 102 undergoes a centerless grinding process to fabricate reduced-diameter core wire portion 110. Various centerless grinding steps may be implemented to achieve a stepped-down taper in core wire portion 110 to thereby incrementally increase its flexibility as it extends distally. In an alternate embodiment, shaft 102 may have a constant diameter proximal shaft region of a harder material, such as cobalt chromium superalloy, stainless steel or titanium, which is connected to a reduced or tapered diameter distal region of a softer more flexible material, such as a softer grade of stainless steel or nitinol. In such an embodiment, the proximal and distal shaft regions may be joined by a coupling sleeve, a weld or solder as would be apparent to one of ordinary skill in the art. In an embodiment, the proximal shaft region may be a hollow tube coupled at its distal end to a proximal end or the distal shaft region. In various embodiments, core wire flat drops 314, 316 may be formed in succession, for example, by stamping a first flat drop, rotating and translating core wire 110 and then stamping the second flat drop. Alternatively, one or more platens, or punch and die sets could be used to concurrently form one or more flat drops.

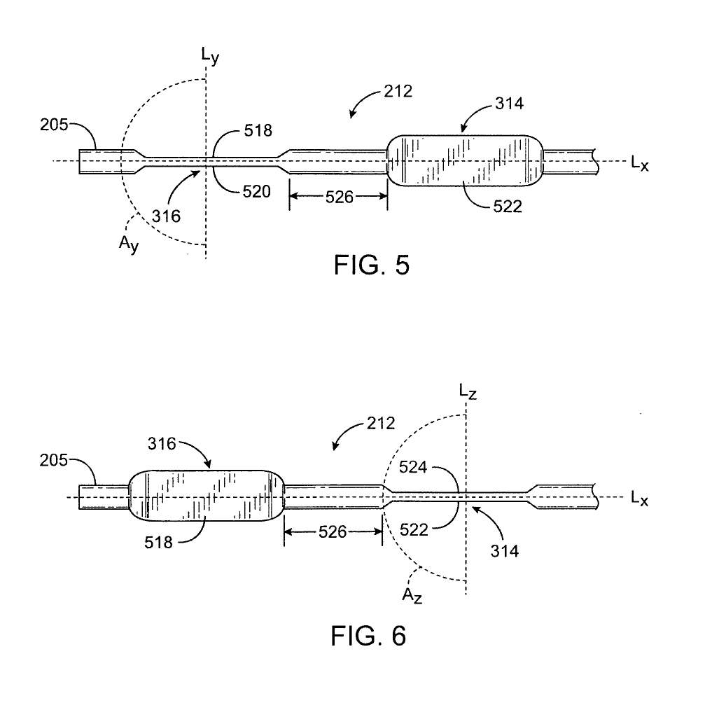

With reference to FIGS. 5 and 6, distal flat drop 316 has a first planar surface 518 substantially in parallel with an opposing second planar surface 520. Similarly, proximal flat drop 314 has a first planar surface 522 substantially in parallel with an opposing second planar surface 524. In the embodiment shown in FIGS. 5 and 6, planar surfaces 518, 520, 522, 524 are substantially parallel to a core wire center axis Lₓ. Planar surfaces 518, 520 of distal flat drop 316 are disposed substantially perpendicular to planar surfaces 522, 524 of distal flat drop 314. In other embodiments, planar surfaces 518, 520 of distal flat drop 316 may be disposed at an angle of less than 90° to planar surfaces 522, 524 of distal flat drop 314. Distal flat drop 316 is axially spaced from proximal flat drop 314 by a linking portion 526, which may be frusto-conical or cylindrical in shape. Length, width and thickness dimensions of proximal and distal flat drops 314, 316 may be the same or different. In an embodiment, a thickness of proximal and/or distal flat drops 314, 316 may taper in a distal direction. In another embodiment, distal flat drop 316 may be wider and/or thinner than proximal flat drop 314.

Proximal flat drop 314 provides increased flexibility of distal tip section 212 for mono-axial bending in a z-direction L_{z} perpendicular to the plane of flat drop 314, as represented by dashed arc line A_{z} in FIG. 6. Similarly, distal flat drop 316 provides increased flexibility of distal tip section 212 for mono-axial bending in a y-direction L_{Y} perpendicular to the plane of flat drop 316, as represented by dashed arc line A_{y} in FIG. 5. Making proximal flat drop 314 and nearby distal flat drop 316 in relatively perpendicular planes increases the bi-axial or omni-axial flexibility of distal tip section 212 of guidewire 100, as compared to a guidewire distal tip segment having a single flat-drop or flat safety/shaping ribbon construction. The axial flexibility of distal tip section 212 may not be perfectly uniform in all directions, however the flexibility in the y- or z-directions L_{Y}, L_{z}, may be only slightly lower than the flexibility in other directions. When distal tip segment 212 is bent in an axial direction other than the y- or z-directions L_{Y}, L_{z}, the bending stress is divided between flat drops 314, 316 such that each flat drop 314, 316 resiliently bends to a degree that is substantially proportional to the extent that each flat drop is aligned with the bend. For example, if distal tip segment 212 is bent only slightly off-axis to y-direction L_{Y}, then distal flat drop 316 will accommodate most of such a deflection, and proximal flat-drop 314 will accommodate only a small portion of such a deflection.

In another embodiment (not shown), an intravascular guidewire according to the present invention may include an elongate shaft having a core wire region with a distal tip segment that includes a plurality of flat drops spaced from each other by short frusto-conical or cylindrical linking portions. Planar surfaces of adjacent flat drops are disposed along the distal tip segment at an angle to each other, such that the surfaces are not in the same plane. As in the previous embodiment, a flexible coil may surround and be attached to at least the core wire region of the guidewire shaft. Further, the core wire region may include a planar surface of at least one flat drop that is substantially perpendicular to a planar surface of at least one other flat drop and/or one or more linking portions that are tapered.

Having a plurality of orthogonal or out-of-plane flat drops in accordance with the disclosure may provide good torque transmission from proximal end 104 to distal tip 205 of guidewire 100, possibly due to the flat drops being axially separated by short linking portion(s) 526. Good torque transmission in core-to-tip construction may enhance the rotational steering or so-called steerability of steerable medical guidewires having small diameters of, e.g., 0.46 mm (0.018 in) or less. Because the flat drops of the disclosure each tend to bend in a single direction, flat drops 314, 316 may be compared to the orthogonal hinges in a conventional Cardan or Hooke's driveshaft universal joint, although flat drops 314, 316 and y- and z-directions L_{Y}, L_{z} are distinctly not within the same plane.

Having a plurality of orthogonal or out-of-plane flat drops in accordance with the disclosure may also increase the rotational strain limit of guidewire 100. Such rotational strain limits are useful design measures for predicting and/or preventing material failure during clinical use, when the guidewire's tip may be trapped while the clinician is rotating the guidewire in an attempt to steer it. A typical bench test for rotational strain limit involves clamping the guidewire distal end in a fixture, and counting the number of rotations of shaft proximal end 104 before material failure, which typically occurs adjacent the guidewire distal end, either in the core wire or in a safety/shaping ribbon, if the device is so equipped. The average number of turns-to-failure in examples made according to the disclosure have been found to exceed the number of turns-to-failure typical of core-to-tip guidewire constructions, and have approached the number of turns-to-failure typical of safety/shaping ribbon constructions.

Having a plurality of orthogonal or out-of-plane flat drops in accordance with this disclosure may also reduce the potential for vessel perforations with the distal end of guidewire 100. As mentioned above, known core-to-tip constructions have tip stiffness suitable for crossing tight stenoses, but such tip stiffness may require additional care to avoid perforating a vessel wall when advancing the guidewire tip through undiseased sections of a patient's vasculature. In comparison to known core-to-tip or single flat-drop constructions, the plurality of out-of-plane flat drops in accordance with this disclosure provide multiple locations for buckling or bending when the guidewire distal tip abuts an obstruction such as a vessel wall. Thus, the instant guidewire disclosure provides embodiments having, in a single device, an improved combination of features not found in known guidewire designs having either core-to-tip, single flat drop, or ribbon-tip constructions.

While various embodiments according to the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

What is claimed is:

1. A medical guidewire comprising:
an elongate shaft having a reduced-diameter distal region that defines a core wire, wherein the core wire has a distal tip segment that includes a proximal flat drop spaced from a distal flat drop by a cylindrical or frusto-conical linking portion; and
a flexible coil surrounding the distal region of the shaft.

2. The guidewire of claim 1, wherein a planar surface of the proximal flat drop is substantially perpendicular to a planar surface of the distal flat drop.

3. The guidewire of claim 1, wherein at least a part of the core wire is tapered.

4. The guidewire of claim 1, wherein the shaft is a unitary structure.

5. The guidewire of claim 1, wherein the shaft distal region is fixedly attached to a proximal region of the shaft.

6. The guidewire of claim 1, wherein the linking portion is tapered.

7. The guidewire of claim 1, wherein a width of the proximal flat drop is not equal to a width of the distal flat drop.

8. The guidewire of claim 7, wherein the width of the proximal flat drop is greater than the width of the distal flat drop.

9. The guidewire of claim 1, wherein a thickness of the proximal flat drop is not equal to a thickness of the distal flat drop.

10. The guidewire of claim 9, wherein the thickness of the proximal flat drop is greater than the thickness of the distal flat drop.

11. The guidewire of claim 1, wherein a thickness of at least one of the proximal and distal flat drops is tapered.

12. An medical guidewire, comprising:
an elongate shaft having a reduced-diameter core wire region, wherein a distal tip section of the core wire portion includes a plurality of flat drops spaced from each other by cylindrical or frusto-conical linking sections; and
a flexible coil surrounding at least the core wire region of the shaft.

13. The guidewire of claim 12, wherein a planar surface of at least one flat drop is substantially perpendicular to a planar surface of at least one other flat drop.
